(19) United States
(12) Patent Application Publication
(10) Pub. No.: US 2023/0338247 A1
(43) Pub. Date: Oct. 26, 2023

(54) WINTERGREEN FLAVOR COMPOSITIONS FREE OF METHYL SALICYLATE

(71) Applicant: Firmenich SA, Satigny (CH)

(72) Inventors: Qing-Bo OUYANG, Plainsboro, NJ (US); Muhammad KIZILBASH, Plainsboro, NJ (US); Bharani ASHOKAN, Plainsboro, NJ (US); Patrice MERCERET, Satigny (CH); Jan Thomas HAINES, Singapore (SG); Yi-Dan TANG, Shanghai (CN); Stuart DASH; Francois GOUYET

(73) Assignee: Firmenich SA, Satigny (CH)

(21) Appl. No.: 18/042,335

(22) PCT Filed: Aug. 13, 2021
(86) PCT No.: PCT/EP2021/072648

§ 371 Date: Feb. 21, 2023

(30) Foreign Application Priority Data
Sep. 22, 2020 (WO) PCT/CN2020/116772
Oct. 14, 2020 (EP) 20201862.8

(51) Int. Cl. A61K 8/34; A61Q 11/00

(52) U.S. Cl. CPC A61K 8/34; A61K 8/347; A61Q 11/00; A61K 2800/28; A61K 2800/30; A61K 2800/92

(57) ABSTRACT

The present disclosure generally provides the flavor compositions that impart a wintergreen taste, but which include reduced amounts methyl salicylate. In some embodiments, the flavor compositions contain no methyl salicylate. In related aspects, the disclosure provides the use of such flavor compositions to reduce or eliminate methyl salicylate in a flavored product, such as a wintergreen-flavored product. In some embodiments, such compositions are incorporated into a flavored product, such as a food product, a beverage product, or an oral care product, such as a toothpaste, a mouthwash, a tooth-whitening composition, mouthwash, chewing gum, and the like.

### CROSS-REFERENCE TO RELATED APPLICATIONS

The present application claims the benefit of priority of PCT Application No. PCT/CN2020/116772, filed Sep. 22, 2020, and European Patent Application No. 20201862.8, filed Oct. 14, 2020, both of which are hereby incorporated by reference as though set forth herein in their entireties.

### TECHNICAL FIELD

The present disclosure generally provides the flavor compositions that impart a wintergreen taste, but which include reduced amounts methyl salicylate. In some embodiments, the flavor compositions contain no methyl salicylate. In related aspects, the disclosure provides the use of such flavor compositions to reduce or eliminate methyl salicylate in a flavored product, such as a wintergreen-flavored product. In some embodiments, such compositions are incorporated into a flavored product, such as a food product, a beverage product, or an oral care product, such as a toothpaste, a mouthwash, a tooth-whitening composition, mouthwash, chewing gum, and the like.

### DESCRIPTION OF RELATED ART

Wintergreen refers to a group of aromatic plants whose extract produces a minty taste. Wintergreen extracts are commonly used in a wide array of products, including mints, chewing gum, candy, smokeless tobacco, dipping tobacco, mouthwash, toothpaste, and American root beer. Natural extracts of wintergreen generally contain an aromatic compound, methyl salicylate. When ingested, methyl salicylate produces salicylic acid as a metabolite, which is the same metabolite produced following the ingestion of aspirin. Thus, natural wintergreen extracts have an effect analogous to a non-steroidal anti-inflammatory drug (NSAID). For this reason, there is a desire to reduce or eliminate the use of compositions containing methyl salicylate from food, beverage, and oral care products.

But many consumers enjoy the wintergreen taste, and have a preference for products having this taste. Therefore, there is a need to develop new flavoring compositions that produce a taste similar to wintergreen extracts, but which benefit from the reduction or elimination of methyl salicylate.

### SUMMARY

The present disclosure relates to the discovery that compositions containing menthol and carvone, and optionally, ethyl benzoate, impart a taste similar to wintergreen without having to include methyl salicylate.

In a first aspect, the disclosure provides flavor compositions comprising menthol and carvone, for example, at a weight-to-weight ratio ranging from 1:2 to 3:1. In some embodiments, the menthol and carvone collectively make up from 15 weight percent to 80 weight percent of the flavor composition. In some embodiments, the flavor composition further comprises ethyl benzoate. In some embodiments, one or more additional ingredients can be present, such as peppermint oil, methyl anthranilate, menthyl lactate, isoamyl acetate, vanillin, p-cresol, eucalyptol, and the like.

In a second aspect, the disclosure provides uses of the flavor composition of the first aspect to reduce or eliminate methyl salicylate in a flavored product, such as a wintergreen-flavored product. In some embodiments, the flavored product is a food product, a beverage product, or an oral care product.

In a third aspect, the disclosure provides methods for reducing or eliminating methyl salicylate in a flavored product (e.g. a wintergreen-flavored product), the method comprising introducing the flavor composition of the first aspect to the flavored product. In some embodiments, the flavored is a food product, a beverage product, or an oral care product.

In a fourth aspect, the disclosure provides flavored products comprising the flavor composition of the first aspect. In some embodiments, the flavored product is free of methyl salicylate. In some embodiments, the flavored is a food product, a beverage product, or an oral care product.

Further aspects, and embodiments thereof, are set forth below in the Detailed Description, the Drawings, the Abstract, and the Claims.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary

A “sweetener”, “sweet flavoring agent”, “sweet flavor entity”, or “sweet compound” herein refers to a compound or ingestibly acceptable salt thereof that elicits a detectable sweet flavor in a subject, e.g., a compound that activates a T1R2/T1R3 receptor in vitro.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Other terms are defined in other portions of this description, even though not included in this subsection.

### Flavor Compositions

In at least one aspect, the disclosure provides flavor compositions comprising menthol. The menthol used in the flavoring composition can have any suitable stereochemical configuration. In some embodiments, the menthol used in the flavoring composition is in the (-) configuration (i.e., the 1R,2S,5R configuration).

In some embodiments, the flavor composition further comprises carvone. The carvone used in the flavoring composition can have any suitable stereochemical configuration, such as the (-) configuration (i.e., R configuration) or the (+) configuration (i.e., S configuration or any mixtures thereof. In some embodiments, the carvone used in the flavoring composition is in the R configuration. In some further embodiments, at least 90% by weight, or at least 95% by weight, or at least 97% by weight, or at least 99% by weight of the carvone present in the flavor composition is in the R configuration, based on the total weight of carvone present in the flavoring composition.

The menthol and carvone can be present in any suitable ratio to each other. For example, in some embodiments, the weight ratio of menthol to carvone in the flavoring composition ranges from 1:4 to 8:1, or from 1:3 to 6:1, or from 1:2 to 3:1, or from 1:1 to 2:1. Carvone and menthol can make up any suitable percentage of the active flavor components of the flavoring composition, namely the flavoring components excluding carriers, such as water, alcohols, and the like. In some embodiments, menthol makes up from 10 weight percent, to 60 weight percent, or from 10 weight percent to 50 weight percent, or from 10 weight percent to 40 weight percent, based on the total weight of active flavoring ingredients in the flavoring composition. In some embodiments, carvone makes up from 5 weight percent, to 60 weight percent, or from 5 weight percent to 50 weight percent, or from 5 weight percent to 40 weight percent, based on the total weight of active flavoring ingredients in the flavoring composition.

In some embodiments, the flavoring composition includes ethyl benzoate. The ethyl benzoate can be present in any suitable ratio relative to menthol. For example, in some embodiments, the weight ratio of menthol to ethyl benzoate in the flavor composition ranges from 1:3 to 8:1, or from 1:2 to 6:1, or from 1:1 to 4:1. Ethyl benzoate can make up any suitable percentage of the active flavor components of the flavoring composition, namely the flavoring components excluding carriers, such as water, alcohols, and the like. In some embodiments, ethyl benzoate makes up from 5 weight percent, to 80 weight percent, or from 10 weight percent to 75 weight percent, or from 15 weight percent to 70 weight percent, based on the total weight of active flavoring ingredients in the flavoring composition.

In some embodiments, the flavoring composition includes methyl benzoate. The methyl benzoate can be present in any suitable ratio relative to menthol. For example, in some embodiments, the weight ratio of menthol to methyl benzoate in the flavor composition ranges from 1:3 to 8:1, or from 1:2 to 6:1, or from 1:1 to 4:1. Methyl benzoate can make up any suitable percentage of the active flavor components of the flavoring composition, namely the flavoring components excluding carriers, such as water, alcohols, and the like. In some embodiments, methyl benzoate makes up from 5 weight percent, to 80 weight percent, or from 10 weight percent to 75 weight percent, or from 15 weight percent to 70 weight percent, based on the total weight of active flavoring ingredients in the flavoring composition.

In some embodiments, the flavoring composition includes peppermint oil. The peppermint oil can be present in any suitable ratio relative to menthol. For example, in some embodiments, the weight ratio of menthol to peppermint oil in the flavor composition ranges from 20:1 to 1:2, or from 12:1 to 1:1, or from 8:1 to 2:1. Peppermint oil can make up any suitable percentage of the active flavor components of the flavoring composition, namely the flavoring components excluding carriers, such as water, alcohols, and the like. In some embodiments, peppermint oil makes up from 2 weight percent to 20 weight percent, or from 2 weight percent to 15 weight percent, or from 2 weight percent to 15 weight percent, based on the total weight of active flavoring ingredients in the flavoring composition.

In some embodiments, the flavoring composition includes methyl anthranilate. The methyl anthranilate can be present in any suitable ratio relative to menthol. For example, in some embodiments, the weight ratio of menthol to methyl anthranilate in the flavor composition ranges from 20:1 to 1:1, or from 12:1 to 1:1, or from 8:1 to 2:1. Methyl anthranilate can make up any suitable percentage of the active flavor components of the flavoring composition, namely the flavoring components excluding carriers, such as water, alcohols, and the like. In some embodiments, methyl anthranilate makes up from 2 weight percent to 10 weight percent, based on the total weight of active flavoring ingredients in the flavoring composition.

In some embodiments, the flavoring composition includes menthyl lactate. The menthyl lactate can be present in any suitable ratio relative to menthol. For example, in some embodiments, the weight ratio of menthol to menthyl lactate in the flavor composition ranges from 80:1 to 2:1, or from 60:1 to 5:1, or from 40:1 to 10:1. Menthyl lactate can make up any suitable percentage of the active flavor components of the flavoring composition, namely the flavoring components excluding carriers, such as water, alcohols, and the like. In some embodiments, menthyl lactate makes up from 0.5 weight percent to 6.0 weight percent, based on the total weight of active flavoring ingredients in the flavoring composition.

In some embodiments, the flavoring composition includes isoamyl acetate. The isoamyl acetate can be present in any suitable ratio relative to menthol. For example, in some embodiments, the weight ratio of menthol to isoamyl acetate in the flavor composition ranges from 400:1 to 5:1, or from 300:1 to 20:1, or from 200:1 to 40:1. Isoamyl acetate can make up any suitable percentage of the active flavor components of the flavoring composition, namely the flavoring components excluding carriers, such as water, alcohols, and the like. In some embodiments, isoamyl acetate makes up from 0.1 weight percent to 2.0 weight percent, based on the total weight of active flavoring ingredients in the flavoring composition.

Other components can be present in flavoring composition, generally at concentrations below 1 weight percent. For example, in some embodiments, vanillin (or vanillin analogs) is present in the flavor composition. In some further such embodiments, vanillin makes up from 700 ppm to 4500 ppm, based on the total weight of active flavoring ingredients in the flavoring composition.

In some embodiments, 2,4-dimethyl acetophenone is present in the flavor composition. In some further such embodiments, 2,4-dimethyl acetophenone makes up from 90 ppm to 350 ppm, based on the total weight of active flavoring ingredients in the flavoring composition.

In some embodiments, α-terpineol is present in the flavor composition. In some further such embodiments, α-terpineol makes up from 1 ppm to 50 ppm, based on the total weight of active flavoring ingredients in the flavoring composition.

In some embodiments, p-cresol is present in the flavor composition. In some further such embodiments, p-cresol makes up from 2000 ppm to 3500 ppm, based on the total weight of active flavoring ingredients in the flavoring composition.

In some embodiments, eucalyptol is present in the flavor composition. In some further such embodiments, eucalyptol makes up from 50 ppm to 6000 ppm, based on the total weight of active flavoring ingredients in the flavoring composition.

In some embodiments, anise oil is present in the flavor composition. In some further such embodiments, anise oil makes up from 5 ppm to 50 ppm, based on the total weight of active flavoring ingredients in the flavoring composition.

In some embodiments, thymol is present in the flavor composition. In some further such embodiments, thymol makes up from 5 ppm to 50 ppm, based on the total weight of active flavoring ingredients in the flavoring composition.

In some embodiments, anisyl acetate is present in the flavor composition. In some further such embodiments, anisyl acetate makes up from 5 ppm to 25 ppm, based on the total weight of active flavoring ingredients in the flavoring composition.

In some embodiments, β-naphthyl ethyl ether is present in the flavor composition. In some further such embodiments, β-naphthyl ethyl ether makes up from 2 ppm to 20 ppm, based on the total weight of active flavoring ingredients in the flavoring composition.

In some embodiments, eugenol is present in the flavor composition. In some further such embodiments, eugenol makes up from 0.5 ppm to 5 ppm, based on the total weight of active flavoring ingredients in the flavoring composition.

In some embodiments, the flavor composition comprises glucosylated natural steviol glycosides (GSGs), for example, to enhance sweetness. As used herein, the term “natural steviol glycosides” refers to steviol glycosides naturally found in the plants of the species Stevia rebaudiana, Stevia phlebophylla, or Rubus chingii. Such compounds are glycosides of the diterpene, steviol. Some non-limiting examples include stevioside, rebaudioside A, rebaudioside C, dulcoside A, and the like. As used herein, the term “glucosylated natural steviol glycosides” refers to compounds obtained by the enzymatic glucosylation of natural steviol glycosides to increase the degree of glucosylation. As used herein, the term “GSG” is used interchangeably with “glucosylated natural steviol glycoside.”

Any suitable GSGs may be used in the various aspects and embodiments disclosed herein. Thus, the GSGs can have any suitable degree of additional glucosylation, and any suitable type of glucosylation. In some embodiments, the GSGs used in the taste-modifying composition have a degree of glucosylation of at least 1.0, meaning that at least one mole of glucose has been enzymatically added to the natural steviol glycosides per mole of natural steviol glycoside. In some other embodiments, the GSGs used in the taste-modifying composition have a degree of glucosylation of at least 1.1, or at least 1.2, or at least 1.3, or at least 1.4, or at least 1.5, or at least 1.6, or at least 1.7, or at least 1.8, or at least 1.9, or at least 2.0, or at least 2.1, or at least 2.2, or at least 2.3, or at least 2.4, or at least 2.5. The glucosylation can be of any suitable type. For example, in some embodiments, the glucose enzymatically added to the natural steviol glycosides is alpha-glucose, beta-glucose, or a combination thereof. In some embodiments, the glucose enzymatically added to the natural steviol glycosides is alpha-glucose. In some embodiments, the glucose enzymatically added to the natural steviol glycosides is beta-glucose. In some embodiments, the glucose enzymatically added to the natural steviol glycosides is a combination of alpha-glucose and beta-glucose. The glucose added by enzymatic glucosylation can be incorporated via any suitable connection. Typically, the added glucose units connect to other glucose glucose units already present on the natural steviol glycoside. In some embodiments, the enzymatically added glucose units are added via 1,6 linkages, meaning that the glucose units add via the 1-position of the added glucose unit and the 6-position of the glucose unit already present on the natural steviol glycoside. In some other embodiments, the enzymatically added glucose units are added via 1,4 linkages. In some other embodiments, the enzymatically added glucose units are added via 1,2 linkages.

The GSGs can be present in the flavor composition at any suitable concentration. In some embodiments, the concentration of GSG in the flavor composition ranges from 1 ppm to 500 ppm, or from 5 ppm to 300 ppm, or from 10 ppm to 200 ppm, or from 20 ppm to 200 ppm, or from 25 ppm to 100 ppm, or from 50 ppm to 200 ppm, or from 50 ppm to 150 ppm, or from 50 ppm to 1000 ppm, based on the total weight of the flavor composition.

The flavor composition can include a variety of other suitable ingredients, such as additional sweeteners, flavorings, carriers, binders, cooling agents, and the like, such as are commonly used in food products, beverage products, or oral care products.

In some embodiments, the flavor composition comprises compounds such as 4-amino-5,6-dimethylthieno[2,3-d]pyrimidin-2(1H)-one, or a salt thereof (such as a hydrochloride salt).

In some embodiments, the flavor composition comprises a lactone. Any suitable lactone or combination of lactones can be used. In some embodiments, the lactone comprises a lactone ring having from 3 to 8 members in the ring, such as a 3-membered lactone ring, a 4-membered lactone ring, a 5-membered lactone ring, a 6-membered lactone ring, a 7-membered lactone ring, or an 8-membered lactone ring.

In some embodiments, the flavor composition comprises a sweetener, such as a low-calorie or zero-calorie sweetener. Examples of suitable sweeteners are set forth in greater detail below. In some embodiments, the sweetener is sucralose, which refers to 1,6-dichloro-1,6-dideoxy-β-D-fructofuranosyl-4-chloro-4-deoxy-α-D-galactopyranoside. In some other embodiments, the sweetener is sodium saccharin. In some further embodiments, the sweetener is xylitol.

For example, in some embodiments, the flavor composition comprises a caloric sugar, such as sucrose, glucose, fructose (e.g., in the form of high-fructose corn syrup), or any combination thereof. In some embodiments, the flavor composition comprises one or more rebaudiosides (such as rebaudioside A, rebaudioside D, rebaudioside E, rebaudioside M, or any combination thereof). In some embodiments, the flavor composition comprises one or more high-intensity artificial sweeteners, such as acefulfame potassium, aspartame, cyclamate, neotame, and the like. In some other embodiments, the flavor composition comprises one or more low-calorie carbohydrates or sugar alcohols, such as allulose, xylitol, erythritol, and the like. In some other embodiments, the flavor composition comprises mogrosides, for example, as monk fruit juice or extract, or as one or more of mogroside III, mogroside IV, mogroside V, siamenoside I, isomogroside V, mogroside IV_{E}, isomogroside IV_{E}, isomogroside IV, mogroside III_{E}, 11-oxomogroside V, the 1,6-alpha isomer of siamenoside I, and any combinations thereof. Additional mogroside compounds that may be suitably included in the flavor composition are described in U.S. Pat. Application Publication No. 2017/0119032.

Various other sweeteners may also be included in the flavor composition. Non-limiting examples include D-psicose, L-ribose, D-tagatose, L-glucose, L-fucose, L-arbinose, D-turanose, D-leucrose, isomalt, lactitol, mannitol, sorbitol, maltodextrin, saccharin, alitame, cyclamic acid, tagatose, maltose, galactose, mannose, lactose, D-tryptophan, glycine, maltitol, lactitol, isomalt, hydrogenated starch hydrolyzate (HSH), chemically modified mogrosides (such as glucosylated mogrosides), carrelame and other guanidine-based sweeteners, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

The flavor composition can, in certain embodiments, comprise any additional ingredients or combination of ingredients as are commonly used in food, beverage, and oral care products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HC1 (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The flavor composition can have any suitable pH when dissolved or suspended in aqueous media, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7.

The flavor composition set forth according to any of the foregoing embodiments, also include, in certain embodiments, one or more additional flavor-modifying compounds, such as compounds that enhance sweetness (e.g., hesperetin, naringenin, neohesperitin, phloretin, rhoifolin, etc.), compounds that block bitterness, compounds that enhance umami, compounds that reduce sourness or licorice taste, compounds that enhance saltiness, compounds that enhance a cooling effect, or any combinations of the foregoing.

In some embodiments, flavor composition disclosed herein comprise one or more sweetness enhancing compounds. Such sweetness enhancing compounds include, but are not limited to, naturally derived compounds, such as hesperitin, naringenin, rhoifolin, glucosylated steviol glycosides, licorice-derived glucuronates, aromadendrin-3-O-acetate, or other like flavonols, or flavonoids, or synthetic compounds, such as any compounds set forth in U.S. Pat. Nos. 8,541,421; 8,815,956; 9,834,544; 8,592,592; 8,877,922; 9,000,054; and 9,000,051, as well as U.S. Pat. Application Publication No. 2017/0119032. Some suitable examples include: 3-((4-amino-2,2-dioxo-1H-benzo[c] [1,2,6]thiadiazin-5-yl)oxy)-2,2-dimethyl-N-propyl-propanamide, N-(1-((4-amino-2,2-dioxo-1H-benzo[c][1,2,6]-thiadiazin-5-yl)oxy)-2-methyl-propan-2-yl)isonicotinamide, or any combination thereof.

In some further embodiments, the flavor composition disclosed herein comprise one or more other umami or kokumi enhancing compounds. Such umami enhancing compounds include, but are not limited to, naturally derived compounds, such as ericamide, or synthetic compounds, such as any compounds set forth in U.S. Pat. Nos. 8,735,081; 8,124,121; and 8,968,708.

In some further embodiments, flavor composition disclosed herein comprise one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727.

In some further embodiments, flavor composition disclosed herein comprise one or more bitterness blocking compounds. Such bitterness blocking compounds include, but are not limited to, naturally derived compounds, such as menthol or analogs thereof, or synthetic compounds, such as any compounds set forth in U.S. Pat. Nos. 8,076,491; 8,445,692; and 9,247,759.

In some further embodiments, flavor composition disclosed herein comprise one or more mouthfeel modifying compounds. Such mouthfeel modifying compounds include, but are not limited to, tannins, cellulosic materials, bamboo powder, and the like.

In some further embodiments, flavor composition disclosed herein comprise one or more flavor masking compounds. Such flavor masking compounds include, but are not limited to, cellulosic materials, materials extracted from fungus, materials extracted from plants, citric acid, carbonic acid (or carbonates), and the like.

The flavor composition disclosed herein can, in certain embodiments, comprise cyclosal.

The flavor composition can be in any suitable form. For example, in some embodiments, the flavor composition is in the form of a solid, such as a solid powder. In some such embodiments, the flavor composition also comprises a solid carrier, such as a carbohydrate. In some other embodiments, the flavor composition is in the form of a liquid solution or suspension. Such a liquid form can be aqueous or non-aqueous, or can also be an emulsion, such as an oil-in-water or water-in-oil emulsion. In some embodiments, the flavor composition is incorporated into a flavored product, such as a food or beverage product, or an oral care product, such as a toothpaste or mouthwash.

In some embodiments, the flavored product or the flavor composition comprise one or more flavorings, or any other additives described in H. Mitchell, SWEETENERS AND SUGAR ALTERNATIVES IN FOOD TECHNOLOGY, Blackwell Publishing Ltd, 2006. As used herein, the term “flavorings” includes those flavors known to the skilled person, such as natural and artificial flavors. These flavorings may be chosen from synthetic flavor oils and flavoring aromatics or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with cooling agents.

Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally, any flavoring or food additive such as those described in CHEMICALS USED IN FOOD PROCESSING, publication 1274, pages 63-258, by the National Academy of Sciences, may be used.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6- dimethyl-5-heptenal, i.e., melonal (melon), 2,6-dimethyloctanal (green fruit), and 2- dodecenal (citrus, mandarin), cherry, grape, strawberry shortcake, and mixtures thereof. These listings of flavorings are merely exemplary and are not meant to limit either the term “flavoring” or the scope of the disclosure generally.

In certain embodiments, mint flavorings, including menthol and related compounds, or other breath-freshening compounds, can be combined with cooling agents, such as those set forth in U.S. Pat. Nos. 9,394,287 and 9,732,071. Additional examples of cooling agents include 5-methyl-2-(propane-2-yl)cyclohexyl-N-ethyloxamate, N-ethyl-p-menthane carboxamide (WS-3, also referred to as menthane-3-carboxylic acid-N-ethyl amide), N-2,3-trimethyl-2-isopropyl butane amide, menthyl lactate, menthone glycerine acetal, mono-menthyl succinate, mono-menthyl glutarate, O-menthyl-glycerine, menthyl-N,N-dimethyl succinamate, N-(4-cyano methyl phenyl)-p-menthane carboxamide, N-(2-(pyridin-2-yl)ethyl)-3-p-menthane carboxamide, menthol and menthol derivatives (e.g., L-menthol, D-menthol, racemic menthol, isomenthol, neoisomenthol, neomenthol) menthyl ether (e.g. (1-menthoxy)-1,2-propanediol, (1-menthoxy)-2-methyl-1,2-propanediol, 1-menthyl-methyl ether), menthyl ester (e.g. menthyl formiate, menthyl acetate, menthyl isobutyrate, menthyl lactates, L-menthyl-L-lactate, L-menthyl-D-lactate, menthyl-(2-methoxy)acetate, menthyl-(2-methoxy ethoxy)acetate, menthyl pyroglutamate), N-(4-cyano methyl phenyl)-p-menthane carboxamide, N-(2-(pyridin-2-yl)ethyl)-3-p-menthane carboxamides, menthyl carbonates (e.g. menthyl propylene glycol carbonate, menthyl ethylene glycol carbonate, menthyl glycerine carbonate or mixtures thereof), menthane carboxylic acid amide (e.g. menthane carboxylic acid-N-ethylamid, N-alpha.-(menthane-carbonyl)glycine ethyl ester, menthane carboxylic acid-N-(4-cyanophenyl)amide, menthane carboxylic acid-N-(alkoxyalkyl)amide), menthone and menthone derivatives (e.g. L-menthone glycerine ketal), 2,3-dimethyl-2-(2-propyl)-butyric acid derivatives (e.g. 2,3-dimethyl-2-(2-propyl)-butyric acid-N-methyl amide), isopulegol or its esters (1-(-)-isopulegol, 1-(-)-isopulegol acetate), menthane derivatives (e.g. p-menthane-3,8-diol), N-(4-cyano methyl phenyl)-p-menthane carboxamides, N-(2-(pyridin-2-yl)ethyl)-3-p-menthane carboxamides, cubebol or synthetic or natural mixtures containing cubebol, pyrrolidone derivates of cycloalkyl dione derivatives (e.g. 3-methyl-2(1-pyrrolidinyl)-2-cyclopentene-1-one) or tetrahydropyrimidine-2-ones (e.g. Icilin or related compounds such as those described in WO 2004/026840), N-(4-cyano methyl phenyl)-p-menthane carboxamide, N-(2-(pyridin-2-yl)ethyl)-3-p-menthane carboxamides, menthyl ether (e.g. (I-menthoxy)-1,2-propanediol, (I-menthoxy)-2-methyl-1,2-propanediol), more polar menthyl esters (e.g. menthyl lactates, L-menthyl-L-lactate, L-menthyl-D-lactate, menthyl-(2-methoxy)acetate, menthyl-(2-methoxy ethoxy)acetate, menthyl pyroglutamate), menthyl carbonates (e.g. menthyl propylene glycol carbonate, menthyl ethylene glycol carbonate, menthyl glycerine carbonate), the semi-esters of menthols with a dicarboxylic acid or the derivatives thereof (e.g. mono-menthyl succinate, mono-menthyl glutarate, mono-menthyl malonate, O-menthyl succinic acid ester-N,N-(dimethyl)amide, O-menthyl succinic acid ester amide), 3,4-methylendioxycinnamic acid-N-cyclohexyl-N-2-pyridylamide, isopropyl-(5-methoxy-2-pyridin-2-yl-pyrimidin-4-yl)-amine, 3,4,6,7,11b,12-hexahydro-3,3-dimethyl-spiro[13H-dibenzo[a,f]quinolizine-1- -3,2′-[1,3]dithiolan]-1(2H)-one, 5,6,10b,11-tetrahydro-3-methyl-spiro[12H-benzo[a]furo[3,4-f]quinolizine-1- -2,2′-[1,3]dithiolan]-1(3H)-one. Most preferred as cooling compounds are compounds selected from the group consisting of 5-methyl-2-(propane-2-yl)cyclohexyl-N-ethyloxamate, N-ethyl-p-menthane carboxamide (WS-3, also referred to as menthane-3-carboxylic acid-N-ethyl amide), menthyl lactate (Frescolat.RTM. ML), menthone glycerine acetal, N-(4-cyano methyl phenyl)-p-menthane carboxamide and (I-menthoxy)-1,2-propanediol.

In some further embodiments, flavor composition disclosed herein comprise one or more cooling agents, such as compounds that modulate TRPM8, such as TRPM8 agonists. Such cooling compounds include, but are not limited to, naturally derived compounds, such as menthol or analogs thereof, or synthetic compounds, such as any compounds set forth in U.S. Pat. Nos. 9,394,287; 9,732,071; 9,840,471; and 10,392,271, as well as compounds such as: N-ethyl-N-(thiophen-2-ylmethyl)-2-(p-tolyloxy)acetamide; N-(1H-pyrazol-3-yl)-N-(thiophen-2-ylmethyl)-2-(p-tolyloxy)acetamide; 2-(4-fluorophenoxy)-N-(1H-pyrazol-3-yl)-N-(thiophen-2-ylmethyl)acetamide; 2-(2-hydroxy-4-methylphenoxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiazol-5-ylmethyl)-acetamide; and 2-((5-methoxy-benzofuran-2-yl)oxy)-N-(1H-pyrazol-3-yl)-N-(thiophen-2-ylmethyl)-acetamide.

In certain aspects, the disclosure provides use of the flavor compositions of any of the foregoing embodiments to reduce or eliminate methyl salicylate in a flavored product, such as a wintergreen-flavored product. In related aspects, the disclosure provides methods of reducing or eliminating methyl salicylate in a flavored product, such as a wintergreen-flavored product, comprising introducing the flavor compositions of any of the foregoing embodiments to the flavored product.

In certain aspects, the disclosure provides flavored products, which comprise flavor compositions of any of the foregoing embodiments. The flavor compositions can be present in any suitable concentration. In some embodiments, the flavor compositions are present in a concentration ranging from 50 ppm to 2 weight percent, depending on the flavored product.

The flavored product can be any flavored product according to the aspects and embodiments set forth below. In some embodiments, the flavored product is a beverage product, such as enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some other embodiments, the flavored product is a food product, such as a packaged meals, canned foods, meal replacement products, and the like. In some embodiments, the flavored product is an oral care product, such as a toothpaste, a mouthwash, or a teeth-whitening composition, such as a composition disclosed in U.S. Pat. Application Pub. No. 2019/0167545, which is incorporated herein by reference.

In some embodiments, the flavored article comprises an alkali metal salt of peroxymonosulfate, such as potassium peroxymonosulfate. In some embodiments, additional salts can also be present in the flavored product. For example, in some embodiments, the flavored product comprises potassium hydrogen sulfate and potassium sulfate.

The flavored product can be in any suitable physical form. For example, in some embodiments, the flavored composition can be in the form of a solid, such as a powder. In a further such embodiment, the flavored product is a toothpowder, such as a toothpowder for whitening teeth, removing stains (e.g., tobacco stains), and the like. In some other embodiments, the flavored product can be a paste or a slurry, such as a tooth paste. In some other embodiments, the flavored product can be in the form of a cream or gel, such as a tooth gel, a skin cream or tooth cream. In some other embodiments, the flavored product can be a liquid suspension or solution, for example, with an aqueous carrier. The flavored product can, in some embodiments, be in the form of an emulsion, such as a microemulsion or nanoemulsion, where a surfactant, emulsifier, and the like can be present. In some embodiments, the flavored product is a personal care, such as a product for application onto skin, face, lips, gums, mouth, and the like. Such personal products can be for any suitable use, such as skin whitening, teeth whitening, and the like.

In certain aspects, the disclosure provides flavored products comprising any compositions of the preceding aspects or embodiments thereof. In some embodiments, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In some embodiments, the flavored product is an oral care product. By “oral care product” is meant a personal care product or other product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition may be in various forms including toothpaste, dentifrice, tooth gel, subgingival gel, mouthrinse, mousse, foam, mouthspray, lozenge, chewable tablet, chewing gum or denture product. The oral care composition may also be incorporated onto strips or films for direct application or attachment to oral surfaces.

In some embodiments, the flavored product is a pharmaceutical product, such as a product designed to carry or deliver certain active pharmaceutical ingredients (APIs). Such APIs can be over-the-counter (OTC) drugs, such as acetaminophen and other OTC cough and cold medications. In some embodiments, the API is a prescription-based drug.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal’s milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children’s breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

In some embodiments, the flavor compositions as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1 - Flavor Compositions

Table 1 and Table 2 set forth sample concentration ranges for different flavor compositions that can suitably reduce or eliminate methyl salicylate in a flavored product.

TABLE 1

| Ingredient | Concentration (wt%) |
|---|---|
| Ethyl Benzoate | 15-70 |
| (-)-Menthol | 10-40 |
| R-(-)-Carvone | 5-40 |
| Peppermint Oil | 2-10 |
| Methyl Anthranilate | 2-10 |
| Menthyl Lactate | 0.5-6 |
| Isoamyl Acetate | 0.1-2 |
| | Concentration (ppm) |
| Vanillin | 700-4500 |
| 2,4-Dimethyl acetophenone | 90-350 |
| α-Terpineol | 5-100 |
| p-Cresol | 2000-3500 |
| Eucalyptol | 50-6000 |
| Anise Oil | 5-50 |
| Thymol | 5-50 |
| Anisyl acetate | 5-25 |
| β-Naphthyl ethyl ether | 2-20 |
| Eugenol | 0.5-5 |

TABLE 2

| Ingredient | Concentration (wt%) |
|---|---|
| (-)-Menthol | 10-60 |
| R-(-)-Carvone | 5-60 |
| Peppermint Oil | 2-20 |
| Methyl Anthranilate | 2-10 |
| Menthyl Lactate | 0.5-6 |
| Isoamyl Acetate | 0.1-2 |
| | Concentration (ppm) |
| Vanillin | 700-4500 |
| 2,4-Dimethyl acetophenone | 90-350 |
| α-Terpineol | 5-100 |
| p-Cresol | 2000-3500 |
| Eucalyptol | 50-6000 |
| Anise Oil | 5-50 |
| Thymol | 5-50 |
| Anisyl acetate | 5-25 |
| β-Naphthyl ethyl ether | 2-20 |
| Eugenol | 0.5-5 |

Tables 3-6 below show some alternative examples of flavor compositions that reduce or eliminate methyl salicylate in wintergreen-flavored products.

TABLE 3

| Ingredient | Flavor 1 | Flavor 2 |
|---|---|---|
| BENZYL ACETATE | 0.50 | 0.50 |
| CYCLOHEXYL ACETATE | 2.05 | 2.05 |
| METHYL ACETATE | 6.00 | 6.00 |
| PIPOL ACETATE | 0.85 | 0.85 |
| STYRALLYL ACETATE | 7.00 | 7.00 |
| ETHYL ACETOACETATE | 7.70 | 7.70 |
| ISOPROPYL ALCOHOL | 5.00 | 5.00 |
| ANETHOL | 3.25 | 3.25 |
| BENZYL BENZOATE | 1.70 | 1.70 |
| METHYL BENZOATE | 2.00 | 2.00 |
| CARVONE LAEVO | 2.20 | 2.20 |
| CITRAL PURE | 0.40 | 0.40 |
| CYCLOHEXYLPROPIONATE D′ALLYLE | 2.00 | 2.00 |
| CYCLOSAL | 0.05 | 0.05 |
| SOLVENT PG (PROPYLENE GLYCOL) | 42.85 | 43.00 |
| ELEMI OIL AR | 0.20 | 0.20 |
| ETHYL FORMATE | 0.40 | 0.40 |
| GLUCOSYLATED STEVIOL GLYCOSIDES | 0.15 | 0 |
| GUAIACOL | 0.20 | 0.20 |
| ACETANISOLE | 0.10 | 0.10 |
| LAVENDER PAYS EO | 0.50 | 0.50 |
| DIETHYL MALONATE | 5.00 | 5.00 |
| MINT SPICATA TERPENLESS SX | 0.05 | 0.05 |
| MINT PIPERITA CANYON SX | 0.10 | 0.10 |
| (+-)-METHYL TRANS-3-OXO-2-PENTYL-1-CYCLOPENTANEACETATE | 9.00 | 9.00 |
| PETITGRAIN PARAGUAY EO | 0.30 | 0.30 |

TABLE 4

| Ingredient | Flavor 3 | Flavor 4 |
|---|---|---|
| EUGENOL F | 2.9 | 4.0 |
| ACETAL | 0.1 | 0.1 |
| THYMOL | 0.1 | 0.1 |
| OREGANO OIL | 0.4 | 0.4 |
| METHYL ANTHRANILATE DIST | 56 | 56 |
| METHYL ETHYLMETHYLANTHRANILATE | 0.4 | 0.4 |
| METHYL BENZOATE | 32.4 | 30.3 |
| ACETOPHENONE | 0.1 | 0.1 |
| MENTHONE PURIFIED | 0.2 | 0.2 |
| ETHYL BENZOATE | 1.0 | 1.0 |
| CINNAMIC ALCOHOL ORD | 4.0 | 5.0 |
| BENZALDEHYDE | 0.2 | 0.2 |
| CINNAMIC ALDEHYDE | 0.6 | 0.6 |
| GUAIACOL | 0.2 | 0.2 |
| ANISYL ALCOHOL | 0.2 | 0.2 |
| MASSOIA LACTONE NAT | 0.8 | 0.8 |
| 2,6-DIMETHYLPYRAZINE | 0.4 | 0.4 |

TABLE 5

| Ingredient | Flavor 5 | Flavor 6 |
|---|---|---|
| BENZYL ACETATE | 1.5 | 1.5 |
| TERPINYL ACETATE FLV | 2.7 | 2.0 |
| ACETOPHENONE | 4.5 | 4.8 |
| CINNAMIC ALCOHOL ORD | 1.2 | 1.2 |
| METHYL ANTHRANILATE DIST | 8 | 8 |
| METHYL BENZOATE | 50 | 49.8 |
| SOLVENT PG | 30 | 30.5 |
| TARRAGON EO | 0.2 | 0.2 |
| EUGENOL F | 0.5 | 0.5 |
| GLYCYRRHIZIN AMMONIA TED LIQ | 0 | 0.4 |
| GUAIACOL | 0.3 | 0.3 |
| PETITGRAIN ITALY EO | 0.3 | 0.3 |
| TERPINENOL 4 | 0.8 | 0.5 |

TABLE 6

| Ingredient | Flavor 7 |
|---|---|
| BENZYL ACETATE | 1.2 |
| DIETHYLENE GLYCOL MONOETHYL ETHER ACETATE | 0.55 |
| TERPINYL ACETATE | 2.0 |
| ACETOPHENONE | 4.0 |
| ACETYL METHYL CARBINOL | 0.15 |
| ANISYL ALCOHOL | 2.7 |
| CINNAMIC ALCOHOL | 0.95 |
| ANETHOL | 1.8 |
| METHYL ANTHRANILATE | 11.9 |
| METHYL BENZOATE | 17.5 |
| DELTA DODECALACTONE | 0.05 |
| DIHYDROESTRAGOLE | 0.50 |
| PROPYLENE GLYCOL | 52.3 |
| ETHYL VANILLIN | 0.10 |
| EUGENOL | 0.40 |
| GUAIACOL | 0.32 |
| ACETANISOLE | 1.20 |
| HELIOTROPINE | 1.4 |
| CIS-3-HEXENYL BENZOATE | 0.10 |
| +/- DIHYDROMINT LACTONE | 0.14 |
| PETITGRAIN OIL | 0.19 |
| TERPINEN-4-OL | 0.55 |

### Example 2 - Flavored Products

Table 7 shows a formulation for a paste dentifrice formulation.

TABLE 7

| Ingredients | Amount (% wt) | Function |
|---|---|---|
| Polyethylene glycol 400 | 2.000 | humectant |
| Xanthan Gum | 0.600 | thickener |
| Sorbitol 70% Solution | 50.000 | humectant |
| Sodium Fluoride | 0.220 | Active ingredient |
| Sodium Benzoate | 0.200 | perservative |
| Water | 15.230 | solvent |
| Hydrated Silica (Tixosil 73) | 22.000 | abrasive |
| Hydrated Silica (Tixosil 43) | 7.000 | thickener |
| Titanium Dioxide CI77891 | 0.500 | opacifier |
| Sodium Lauryl Sulfate | 1.250 | surfactant |
| Flavor | 1.200 | Flavoring agent |
| Sodium saccharin | 0.25 | sweetener |

Table 8 shows a formulation for a gel dentifrice formulation.

TABLE 8

| Ingredients | Amount (% wt) | Function |
|---|---|---|
| Xanthan Gum | 0.800% | thickener |
| Flavor | 1.200% | Flavoring agent |
| DI/Purified Water | Q.S to Final Wt. | solvent |
| Sodium Lauryl Sulfate | 1.500% | surfactant |
| Sodium Fluoride | 0.220% | Active ingredient |
| Sorbitol 70% Solution | 30.000% | humectant |
| Sodium Saccharin | 0.200% | sweetener |
| Glycerine | 5.000% | humectant |
| Sodium Benzoate | 0.200% | preservative |
| Sucralose | 0.050% | sweetener |
| Hydrated Silica (Tixosil 63) | 20.000% | abrasive |
| Hydrated Silica (Tixosil 43) | 5.000% | thickener |

Table 9 shows a formulation for a chalk base formulation.

TABLE 9

| Ingredients | Amount (% wt) | Function |
|---|---|---|
| water | Q.S to Final Wt. | Solvent |
| sorbitol | 26 | Humectant |
| Methylparaben | 0.2 | Preservative |
| Xanthan Gum | 0.6 | Thickener |
| Polyethylene glycol 400 | 1.6 | humectant |
| Hydrated Silica | 10 | thickener |
| Calcium carbonate | 28 | Abrasive |
| Sodium Lauryl Sulfate | 1.25 | Surfactant |
| Flavor | 1 | Flavoring agent |
| Sodium Saccharin | 0.2 | sweetener |

Table 10 shows a formulation for a calcium phosphate dihydrate toothpaste base.

TABLE 10

| Ingredients | Amount (% wt) | Function |
|---|---|---|
| Polyethylene glycol | 2 | Humectant |
| Binder | 1 | Thickener |
| Sorbitol 70% Syrup | 40 | Humectant |
| Glycerine | 10 | Thickener |
| Sodium Fluoride | 0.1 | Active ingredient |
| Sodium monofluorophosphate | 0.5 | Active ingredient |
| Methyl paraben | 0.1 | Preservative |
| Propyl paraben | 0.1 | Preservative |
| Dicalcium phosphate Dihydrate | 40 | Abrasive |
| Titanium Dioxide CI77891 | 0.5 | Opacifier |
| Sodium Lauryl Sulfate | 2 | Surfactant |
| Flavor | 1.2 | Flavoring agent |
| Sodium Saccharin | 0.2 | sweetener |
| water | 2.4 | Solvent |

Table 11 shows a formulation for an alcohol-free mouthwash formulation.

TABLE 11

| Ingredients | Amount (% wt) | Function |
|---|---|---|
| Propylene Glycol | 10.000 | humectant |
| Flavor | 0.240 | Flavouring agent |
| DI/Purified Water | Q.S to Final Wt. | solvent |
| Poloxamer 407 NF | 0.240 | Co-solvent |
| Sodium Lauryl Sulfate | 0.040 | Surfactant |
| Sorbitol 70% Solution | 10.000 | humectant |
| Sodium Saccharin | 0.030 | sweetener |
| Glycerine | 3.000 | humectant |
| Sodium Benzoate | 0.100 | preservative |
| Sucralose | 0.020 | sweetener |
| Benzoic Acid | 0.050 | preservative |

Table 12 shows a formulation for an alcohol-containing mouthwash formulation.

TABLE 12

| Ingredients | Amount (% wt) | Function |
|---|---|---|
| Ethyl Alcohol 190 Proof | 15.000% | solubilizer |
| Flavor | 0.240% | Flavoring agent |
| DI/Purified Water | Q.S to Final Wt. | solvent |
| Poloxamer 407 NF | 0.240% | Co-solvent |
| Sodium Lauryl Sulfate | 0.040% | Surfactant |
| Sorbitol 70% Solution | 10.000% | humectant |
| Sodium Saccharin | 0.030% | sweetener |
| Glycerine | 3.000% | humectant |
| Sodium Benzoate | 0.100% | preservative |
| Sucralose | 0.020% | sweetener |
| Benzoic Acid | 0.050% | preservative |

### Example 3 - Performance Testing

A control flavor formulation (C) and six test examples (E1-E6) were prepared. The flavor is intended to flavor a toothpaste. Table 13 shows the composition of the control and the six test samples.

TABLE 13

| Ingredient | C | E1 | E2 | E3 | E4 | E5 | E6 |
|---|---|---|---|---|---|---|---|
| Gel base | 98.25% | 98.25% | 98.25% | 98.25% | 98.25% | 98.25% | 98.25% |
| Mint Avensis | 0.10% | 0.10% | 0.10% | 0.10% | 0.10% | 0.10% | 0.10% |
| Mint Piperita Cascade | 0.10% | 0.10% | 0.10% | 0.10% | 0.10% | 0.10% | 0.10% |
| Menthol | 0.40% | 0.40% | 0.40% | 0.40% | 0.40% | 0.40% | 0.40% |
| Eugenol | 0.10% | 0.10% | 0.10% | 0.10% | 0.10% | 0.10% | 0.10% |
| Anethol | 0.10% | 0.10% | 0.10% | 0.10% | 0.10% | 0.10% | 0.10% |
| Methyl Salicylate | 0.20% | 0.00% | 0.00% | 0.00% | 0.00% | 0.00% | 0.00% |
| Flavor 1 | 0.00% | 0.03% | 0.00% | 0.00% | 0.00% | 0.00% | 0.00% |
| Flavor 2 | 0.00% | 0.00% | 0.03% | 0.00% | 0.00% | 0.00% | 0.00% |
| Flavor 3 | 0.00% | 0.00% | 0.00% | 0.20% | 0.00% | 0.00% | 0.00% |
| Flavor 4 | 0.00% | 0.00% | 0.00% | 0.00% | 0.20% | 0.00% | 0.00% |
| Flavor 5 | 0.00% | 0.00% | 0.00% | 0.00% | 0.00% | 0.18% | 0.00% |
| Flavor 6 | 0.00% | 0.00% | 0.00% | 0.00% | 0.00% | 0.00% | 0.18% |
| Sodium Saccharin | 0.25% | 0.25% | 0.25% | 0.25% | 0.25% | 0.25% | 0.25% |
| Purified Water | 0.50% | 0.67% | 0.67% | 0.50% | 0.50% | 0.52% | 0.52% |

### Step 1: Smell Evaluation

Rinse the toothbrush under running water for 5 secs to moisten and soften the bristles
Take 2 g of sample of toothbrush.
Bring toothbrush (from above) to 1″ below nose & smell mouthwash .
Measure the “Overall Aroma Intensity” on the normal intensity scale (0=none to 5=extremely high)

### Step 2: Evaluation on Brushing

Brush around the teeth for 1 min, minimizing brushing of gums
Expectorate and rinse with water (2-3 times)
Make use of the plain milk and rice puffs for palette cleaning
Keep the gap of minimum 20 mins between each tasting samples
Evaluate all products in 1 session, repeat in subsequent session to get replicates

All the prototypes were compared against the control sample. Rank 1 means the most close to the control samples and Rank 3 means the worst close to the control sample. Sample size: 15. Table 14 shows the results, giving the number of times that each of the test samples was in the top 3, and the number of times it scored Rank 1, Rank 2, and Rank 3.

TABLE 14

| | Top 3 | Rank 1 | Rank 2 | Rank 3 |
|---|---|---|---|---|
| E1 | 5 | 3 | 1 | 1 |
| E2 | 4 | 1 | 2 | 1 |
| E3 | 9 | 2 | 4 | 3 |
| E4 | 4 | 1 | 2 | 1 |
| E5 | 8 | 2 | 2 | 4 |
| E6 | 7 | 4 | 2 | 1 |

A control flavor formulation (C) and six additional test examples (E7-E12) were prepared. The flavor is intended to flavor a mouthwash. Table 15 shows the composition of the control and the six test samples.

TABLE 15

| Ingredient | C | E7 | E8 | E9 | E10 | E11 | E12 |
|---|---|---|---|---|---|---|---|
| Ethanol | 23.0% | 23.0% | 23.0% | 23.0% | 23.0% | 23.0% | 23.0% |
| Thymol | 0.0600% | 0.0600% | 0.0600% | 0.0600% | 0.0600% | 0.0600% | 0.0600% |
| Menthol | 0.0400% | 0.0400% | 0.0400% | 0.0400% | 0.0400% | 0.0400% | 0.0400% |
| Eucalyptol | 0.0900% | 0.0900% | 0.0900% | 0.0900% | 0.0900% | 0.0900% | 0.0900% |
| Methyl Salicylate | 0.0647% | 0.0000% | 0.0000% | 0.0000% | 0.0000% | 0.0000% | 0.0000% |
| Flavor 1 | 0.0000% | 0.0100% | 0.0000% | 0.0000% | 0.0000% | 0.0000% | 0.0000% |
| Flavor 2 | 0.0000% | 0.0000% | 0.0100% | 0.0000% | 0.0000% | 0.0000% | 0.0000% |
| Flavor 3 | 0.0000% | 0.0000% | 0.0000% | 0.0647% | 0.0000% | 0.0000% | 0.0000% |
| Flavor 4 | 0.0000% | 0.0000% | 0.0000% | 0.0000% | 0.0647% | 0.0000% | 0.0000% |
| Flavor 5 | 0.0000% | 0.0000% | 0.0000% | 0.0000% | 0.0000% | 0.0582% | 0.0000% |
| Flavor 6 | 0.0000% | 0.0000% | 0.0000% | 0.0000% | 0.0000% | 0.0000% | 0.0582% |
| Poloxamer 407 | 0.2100% | 0.2100% | 0.2100% | 0.2100% | 0.2100% | 0.2100% | 0.2100% |
| Benzoic Acid | 0.0900% | 0.0900% | 0.0900% | 0.0900% | 0.0900% | 0.0900% | 0.0900% |
| Sodium Benzoate | 0.0500% | 0.0500% | 0.0500% | 0.0500% | 0.0500% | 0.0500% | 0.0500% |
| Water | 76.39% | 76.45% | 76.45% | 76.39% | 76.39% | 76.40% | 76.40% |

### Step 1: Smell Evaluation

Pour 20 ml/as mentioned on the pack of product in a cup .
Bring shot cup (from above) to 1″ below nose & smell mouthwash .
Measure the “Overall Aroma Intensity” on the normal intensity scale (0=none to 5=extremely high) .

### Step 2: Evaluation on Swishing

Take the above sample; swish for 30 seconds & expectorate .
Measure all attributes “immediately after swishing” on the normal intensity scale (0=none to 5=extremely high)
Make use of plain milk and rice puffs for palette cleaning.
Give a gap of minimum 30 minutes between consecutive samples.
Evaluate all products in 1 session, repeat in subsequent session to get replicates

All the prototypes were compared against the control sample. Rank 1 means the most close to the control samples and Rank 3 means the worst close to the control sample. Sample size: 15. Table 16 shows the results, giving the number of times that each of the test samples was in the top 3, and the number of times it scored Rank 1, Rank 2, and Rank 3.

TABLE 16

| | Top 3 | Rank 1 | Rank 2 | Rank 3 |
|---|---|---|---|---|
| E7 | 7 | 0 | 5 | 2 |
| E8 | 8 | 3 | 3 | 2 |
| E9 | 11 | 4 | 5 | 2 |
| E10 | 7 | 5 | 2 | 0 |
| E11 | 3 | 1 | 1 | 1 |
| E12 | 7 | 2 | 0 | 5 |

1. A flavor composition for simulating a wintergreen taste, the composition comprising
(-)-menthol; and
one or more additional ingredients selected from the group consisting of R-(-)-carvone, eugenol, thymol, ethyl benzoate, methyl benzoate, and combinations thereof.

2. The flavor composition of claim 1, wherein the composition is substantially free of methyl salicylate.

3. The flavor composition of claim 2, wherein the composition comprises no more than 50 ppm, or no more than 20 ppm, or no more than 10 ppm, or no more than 5 ppm, or no more than 1 ppm of methyl salicylate.

4. A method of reducing or eliminating methyl salicylate in a wintergreen-flavored product, the method comprising introducing the flavor composition of claim 1 to the flavored product.

5. The method of claim 4, wherein the wintergreen-flavored product is an oral care product.

6. A flavored product, which comprises the flavor composition of claim 1.

7. The flavored product of claim 6, wherein the flavored product is an oral care product.
